(19) United States
(12) Patent Application Publication
(10) Pub. No.: US 2026/0041476 A1
(43) Pub. Date: Feb. 12, 2026

(54) CRYOABLATION PROBES WITH HEAT EXCHANGER IN RETURN CRYOGEN LINE

(71) Applicant: Varian Medical Systems, Inc., Palo Alto, CA (US)

(72) Inventor: Ryan Medema, Georgetown, TX (US)

(21) Appl. No.: 18/800,921

(22) Filed: Aug. 12, 2024

(51) Int. Cl. A61B 18/02; A61B 18/00

(52) U.S. Cl. CPC A61B 18/0218; A61B 2018/00041; A61B 2018/0231

(57) ABSTRACT

A cryoprobe assembly includes a needle, a flexible conduit, and a handle portion configured to couple the needle to the flexible conduit to create a cryogen flow path. The flexible conduit includes a counterflow heat exchanger to transfer thermal energy from supply cryogen to return cryogen prior to reaching the needle in order to achieve a predetermined temperature during Joule-Thompson expansion in the needle.

### FIELD

The present disclosure relates to cryoablation probes that include improved heat exchange between supply cryogen and return cryogen.

### BACKGROUND

This section provides background information related to the present disclosure which is not necessarily prior art.

Systems and methods for providing cryoablation treatments may include cryoablation probes that are introduced at or near target tissue in a patient. A cryoablation system may include an extremely cold cryogen (liquid, gas, or mixed phase) that may be passed through a probe in thermal contact with the target tissue. Heat from the tissue passes from the tissue, through the probe, and into the cryogen that removes heat from the targeted tissue. This removal of heat causes tissue to freeze, resulting in the destruction of the targeted tissue. It is desirable that the cryogen is of sufficiently low temperature to quickly and efficiently cause the targeted tissues to freeze.

Traditional or existing cryoprobes and related structures often include inefficient structures and/or bulky structures to transport the cryogen from a cryogen source to the cryoprobe that is inserted at or near the target tissue. Such existing cryoprobes and related structures may be difficult to manufacture and/or may be larger than desired in order to include the structure necessary to achieve sufficiently low temperatures at the target tissue. In addition, existing cryoprobes and related structures may be not be easily manipulated and/or may not be easily maintained in a desired location due to their size or mass. There exists a need, therefore, for improved cryoprobes and related structures that efficiently achieve a desired temperature of the cryogen at the target tissue and are of suitable size to be easily manipulated, retained at a desired a position while being manufactured at low cost.

### SUMMARY

This section provides a general summary of the disclosure, and is not a comprehensive disclosure of its full scope or all of its features.

In various embodiments of the present disclosure, improved cryoablation assemblies and cryoprobes are provided. The cryoprobes of the present disclosure may include heat exchangers incorporated into the flexible lines that transfer cryogen to and from a cryogen source. The cryogen lines of the present disclosure may include various structures that may improve the transfer of thermal energy from the supply cryogen to the return cryogen. Such heat exchangers may lower the temperature of the high pressure cryogen that is supplied to the needle of the cryoprobe where it may undergo Joule-Thompson expansion that drops the temperature of the needle to perform a cryoablation treatment.

In some embodiments of the present disclosure, a cryoprobe assembly may include a needle, a flexible conduit, and a handle portion configured to couple the needle to the flexible conduit to create a cryogen flow path. The flexible conduit may include a counterflow heat exchanger.

In one aspect, the handle portion does not include a heat exchanger.

In another aspect, the needle defines a Joule-Thompson expansion chamber.

In another aspect, the counterflow heat exchanger is flexible.

In another aspect, the counterflow heat exchanger has a length of at least 12 inches.

In another aspect, the flexible conduit includes a plurality of counter flow heat exchangers spaced apart from one another.

In another aspect, the handle portion includes a vacuum chamber to insulate an exterior of the handle portion from a cryogen flow path.

In another aspect, the counterflow heat exchanger includes a supply path through which cryogen flows toward a distal end of the needle and a return path through which the cryogen flows away from the distal end of the needle. The counterflow heat exchanger is configured to transfer thermal energy from the cryogen in the supply path to the cryogen in the return path to lower a temperature of the cryogen in the supply path.

In another aspect, the counterflow heat exchanger has a predetermined length that causes the distal end of the needle to achieve a temperature of about -140 degrees Celsius or lower.

In another aspect, the supply path or the return path of the counterflow heat exchanger has a helical shape.

In another aspect, the counterflow heat exchanger includes a plurality of fins extend from the supply path into the return path.

In another aspect, the handle portion is configured to transition from a first axial direction of the flexible conduit to a second axial direction of the needle.

In another aspect, the second axial direction is offset 90 degrees from the first axial direction.

In another aspect, the handle portion is adjustable to vary an offset angle between the first axial direction and the second axial direction.

In another aspect, a maximum width of the handle portion is less than about 10 mm.

In another aspect, a maximum width of the handle portion is less than about 5 mm.

In another aspect, a mass of the handle portion is less than about X grams.

In some embodiments of the present disclosure, a method of providing cryogen to a cryoprobe is provided. The method may include moving a cryogen through a supply path in a flexible conduit toward a distal end of a needle of a cryoprobe and pre-cooling the cryogen in the flexible conduit prior to reaching the distal end of the needle of the cryoprobe. The method may also include allowing the pre-cooled cryogen to expand in a Joule-Thompson expansion chamber at the distal end of the needle to achieve a temperature of about -140 degrees Celsius or lower.

In one aspect, the pre-cooling of the cryogen is performed using a counterflow heat exchanger positioned in the flexible conduit.

In another aspect, the step of moving the cryogen through the supply path includes moving the cryogen through a handle portion of the cryoprobe that connects the flexible conduit to the needle, and the step of pre-cooling the cryogen is performed without a heat exchanger in the handle portion.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an illustration of an example cryoablation apparatus that may be used diagram illustrating an example cryoablation system in accordance with some embodiments of the present disclosure.

FIG. 2 is an illustration showing an example cryoprobe assembly that includes a cryogen line with heat exchanger in accordance with some embodiments of the present disclosure.

FIG. 3 is a side cross-sectional view of an example cryogen line in accordance with some embodiments of the present disclosure.

FIG. 4 is a side cross-sectional view of another example cryogen line in accordance with some embodiments of the present disclosure.

FIG. 5 is a side cross-sectional view of another example cryogen line in accordance with some embodiments of the present disclosure.

FIG. 6 is an end cross-sectional view of an example cryogen line in accordance with some embodiments of the present disclosure.

FIG. 7 is an end cross-sectional view of another example cryogen line in accordance with some embodiments of the present disclosure.

FIG. 8 is an end cross-sectional view of another example cryogen line in accordance with some embodiments of the present disclosure.

FIG. 9 is a graph showing performance of an example cryoprobe assembly of the present disclosure compared to traditional cryoprobe assemblies showing a temperature of a needle during freezing cycles.

FIG. 10 is a graph showing temperature of supply and return cryogen in a traditional cryogen line.

FIG. 11 is a graph showing temperature of supply and return cryogen in an example cryogen line of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms "a,” "an," and "the" may be intended to include the plural forms as well, unless the context clearly indicates otherwise. The terms "comprises," "comprising," “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being "on," “engaged to,” "connected to," or "coupled to" another element or layer, it may be directly on, engaged, connected or coupled to the other element or layer, or intervening elements or layers may be present. In contrast, when an element is referred to as being "directly on," “directly engaged to,” "directly connected to," or "directly coupled to" another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term "and/or" includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” "beneath," "below," "lower," "above," "upper," and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as "below" or "beneath" other elements or features would then be oriented "above" the other elements or features. Thus, the example term "below" can encompass both an orientation of above and below. The device may be otherwise oriented (rotated 90 degrees or at other orientations) and the spatially relative descriptors used herein interpreted accordingly.

In some embodiments of the present disclosure, a cryoprobe assembly is provided that includes a heat exchanger positioned in or integrated into the cryogen return line of the assembly. The heat exchanger may have a length of more than 12 inches in some examples. In other examples, the heat exchange may extend along an entire length of the return line from the handle and/or needle to the cryogen source. Thus, the heat exchanger may be as long as 12 feet in some examples. By locating a heat exchanger in the cryogen return line, a heat exchanger that may otherwise be included in a handle of the cryoprobe may be eliminated. This, in turn, may allow the cryoprobe and/or handle to have a smaller size, a smaller profile, and a smaller mass than existing cryoprobes. In addition, the length of the heat exchanger is not limited by the size or length of the handle of the cryoprobe. This may result in improved usability in the clinical environment and may improve the performance of the cryoprobe.

Cryoprobes may be configured to utilize the Joule-Thompson effect to significantly decrease the operating temperature of the tip of the cryoprobe. As explained above, the tip of the cryoprobe may be inserted at or near a target tissue during a cryoablation procedure. A cryogen is passed through the cryoprobe and allowed to remove heat from the target tissue. Due to the low temperatures of the cryogen, the target tissue at or near the tip of the cryoprobe freezes destroying the target tissue. It is desirable to freeze the target tissue quickly so as to reduce a likelihood of harm to healthy tissue and to reduce a length of cryoablation treatments.

In Joule-Thompson cryoprobes, the cryogen is transferred to the tip of the cryoprobe at a high pressure. In some instances, the pressure may be at or above 3000 pounds per square inch (psi). In other examples, the pressure may be at other elevated pressures. This high pressure cryogen is allowed to expand in a chamber in the tip of the cryoprobe whereby the drop in pressure is accompanied by a drop in temperature. The Joule-Thompson effect allows the temperature of the cryogen at the tip of the cryoprobe to drop to temperatures less than -100 degrees Celsius. It may be desirable to achieve even lower temperatures than -100 degrees Celsius such as temperatures lower than -120 degrees Celsius or -130 degrees Celsius.

To achieve these lower temperatures, existing cryoprobe assemblies may include a heat exchanger in the handle of the cryoprobe assembly whereby the returning cryogen that is at a low temperature after the Joule-Thompson expansion, is used to cool the incoming supply cryogen that is at a high temperature. With the heat exchanger located in the handle, existing cryoprobes may achieve temperatures in the range of -120 degrees Celsius to -130 degrees Celsius.

These existing cryoprobes, however, suffer from drawback and/or limitations. The cryoprobes may be limited in the amount of heat transfer that can be accomplished between the supply cryogen and the return cryogen because the heat exchanger is located in the handle of the cryoprobe. The heat exchanger may also cause the handle to be of a larger size than needed to accomplish the required amount of heat exchange. It is desirable to have handles of small size or limited size because of considerations in the clinical environment.

In the clinical environment, a cryoablation treatment may be performed in a room in which the patient is positioned on a table or gantry that is moved into and out of an imaging device such as a Computed Tomography (CT) device or MRI device. The space in such imaging devices are limited. In an example treatment, the needle of the cryoprobe may be inserted into a patient at or near the target tissue in the patient. To confirm positioning of the needle and/or to collect other treatment information, the patient may be transferred into and out of the imaging device with the cryoprobes inserted at the target tissue. The cryoprobes may have a right angle bend at the handle to allow the cryogen supply/return lines to be routed in a manner to allow the patient to be moved into and out of the imaging device.

It is undesirable that the needles of the cryoprobes move or are disturbed after the needles are positioned at the target tissue. The handles of the cryoprobes may be of a size or have a mass that may cause or lead to movement of the needle. Furthermore, if the handle of the cryoprobe is too large, the handle may obstruct movement of the patient into or out of the imaging device. It may be desirable, therefore, to reduce or minimize a size of the handle of the cryoprobe. Such reduction in size may make it easier to move the patient without movement of or disturbance to the needle of the cryoprobes during treatment.

It is also noted that in some treatments multiple needles are inserted at or near the target tissue. It may be desirable to position the needles close to each other to freeze the target tissue. The handles of existing cryoprobes can be large enough or have widths or diameters that can interfere with the positioning of cryoprobe needles close to one another.

The cryoprobe assemblies of the present disclosure are improvements over existing cryoprobes because the handles of the cryoprobes of the present disclosure can be smaller (i.e., have smaller widths, smaller lengths, smaller outer diameters, smaller overall mass) than existing cryoprobes. Due to the reduced size, the cryoprobes of the present disclosure less likely to move or be disturbed during a treatment. In addition, the reduced size may allow needles to be positioned closer together without interference. Still further, the cryoprobes of the present disclosure may achieve lower temperatures more quickly than existing cryoprobes because of improved heat exchange between the supply cryogen and the return cryogen.

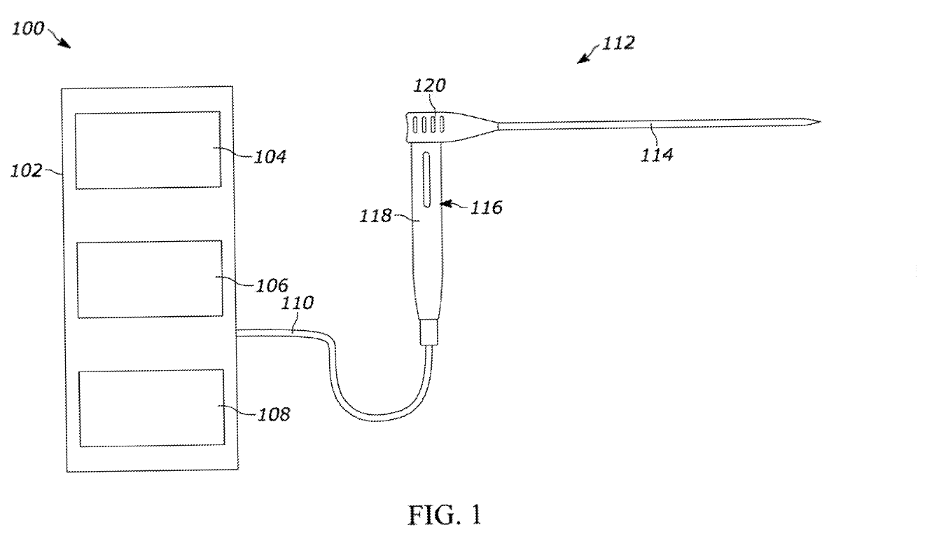

Referring now to FIG. 1, an example cryoablation apparatus 100 is shown. The cryoablation apparatus 100 may include a cryoablation console 102, and a cryoprobe assembly 112. The cryoablation console 102 may include a cryo-controller 104, a cryogen delivery apparatus 106 and a cryogen source 108. The cryo-controller 104 may include a computing device or other controller that can be used to control delivery of a cryogen (e.g., Argon, helium, Nitrogen, or the like) from the cryogen source 108 to the cryoprobe assembly 112 using the cryogen delivery apparatus 106. The cryogen source 108 may be a suitable Dewar or other container that can be filled with a cryogen. The cryogen delivery apparatus 106 may include a pump, one or more valves, and other suitable fluid delivery devices to fluidly connect the cryogen source to the cryogen line 110 of the cryoprobe assembly 112. Upon the initiation of a freezing cycle, the cryo-controller 104 may cause the cryogen to be moved through a cryogen flow path that includes a cryogen supply line from the cryogen source through the cryogen line 110 to the cryoprobe 114. The cryogen may then flow back to the cryogen source via a cryogen return line from the cryoprobe 114 back to the cryogen source 108.

The cryogen line 110 may be a flexible tube or other conduit that may include multiple lumens to allow the cryogen to flow in a supply direction to the cryoprobe 114 and separately in a return direction away from the cryoprobe 114. The cryogen line 110 may of sufficient length to allow the console 102 to be positioned near the patient in a treatment room and to allow the patient to be moved into and out of an imaging device. In some examples, the cryogen line may be at least about 12 feet in length. In other examples, the cryogen line 110 may have other lengths.

The cryogen line 110 fluidly couples the cryogen source 108 to the cryoprobe 114. The cryoprobe 114 may be a needle or other elongated member that is configured to be inserted into patient tissue and be positioned at or near the target tissue during treatment. The cryoprobe 114 may be configured as a cylindrical needle having an outer diameter in a range of about 1 mm to about 4 mm. The cryoprobe 114 may also include a handle 116. The handle 116 may be configured with a first portion 118 and a second portion 120. The first portion 118 may be substantially aligned with the cryogen line 110 and the second portion 120 may offset at an angle relative to the first portion 118. The offset angle between the first portion 118 and the second portion 119 may be about 90 degrees to define a right angle handle. In other example, the first portion 118 and the second portion 120 may be offset at different angles.

In some examples, the handle 116 may include a vacuum chamber positioned at or near an outside surface of the handle 116. The vacuum chamber may insulate the exterior of the handle from the extremely low operating temperatures of the cryogen that moves through the handle to the cryoprobe 114. This may allow an operator to touch or otherwise manipulate the cryoprobe 114 during treatment.

While not shown, more than one cryoprobe assembly 112 may be coupled to the console 102. Multiple cryoprobe assemblies 112 may be used during a single cryoablation treatment in combination. The console 102 may be configured to deliver cryogen to the multiple cryoprobe assemblies 112. The cryoprobes 114 of each cryoprobe assembly 112 may be similar to reach other or may be different to produce iceballs of different sizes and shapes so as to freeze and destroy the target tissue.

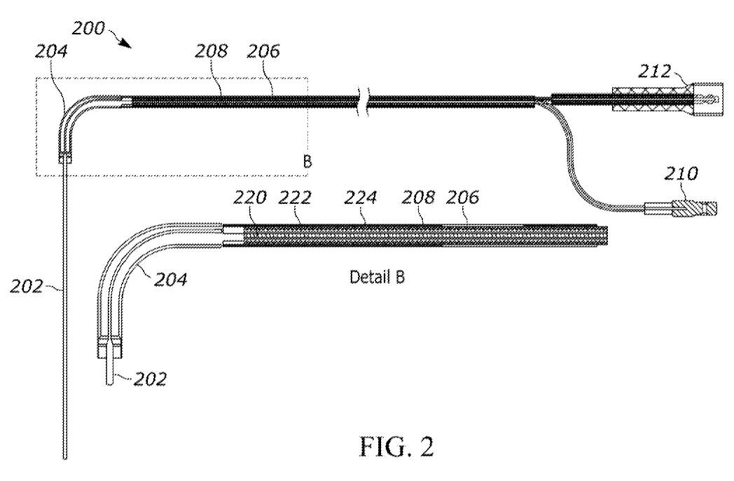

Referring now to FIG. 2, an example cryoprobe assembly 200 is shown. The cryoprobe assembly 200 may be used in combination with the cryoablation apparatus 100 previously described. In this example, the cryoprobe assembly 200 may include a needle 202, a handle 204, a cryogen line 208, a sensor assembly 210, and a connector 212. The cryoprobe assembly 200 may be coupled to a cryogen source using the connector 212. The connector 212 may fluidly connect and seal the cryoprobe assembly 200 to the cryogen source and allow cryogen to be supplied to the cryoprobe and cryogen to be returned to the cryogen source. The sensor assembly 210 may include a temperature, pressure, or other sensor and may be coupled to the console 102 to provide information regarding the operating parameters of the cryoprobe assembly 200.

The needle 202 may be any suitable cryoablation needle and may extend in an axial direction away from handle 204. The needle 202 may be made of suitable metal or alloy such as stainless steel. In other examples, other suitable materials such as ceramic may be used. The needle 202 may have various sizes or diameters that may be suitable to produce a freezing zone or iceball that may be desired to freeze the target tissue. In various examples, the needle 202 may have an outer diameter in a range of about 1 mm to about 4 mm. In other examples, other suitable sizes may be used.

The needle 202 may include an outer shell and an inner lumen that extends radially along its length. The outer shell and the inner lumen may be positioned concentrically with each other. The inner lumen may include an opening at one end toward a tip 228 of the needle. During a freezing cycle, the cryogen (at high pressure) may be passed through the inner lumen toward the tip 228 and the cryogen may exit the inner lumen and allowed to expand in a Joule-Thompson chamber at the tip 228. Such expansion causes the temperature of the cryogen to drop. The cryogen may then flow away from the tip 228 through a return pathway defined as a space between an inner surface of the outer shell and the outer surface of the inner lumen.

The needle 202 may be coupled to the handle 204. The handle may be formed of plastic, composite, or other suitable material. The handle 204 may be fluidly and mechanically coupled to the needle 202. The inner lumen and the outer shell of the needle 202 may be fluidly coupled to a supply lumen and a return pathway, respectively. In this manner, the supply cryogen may be supplied to the needle through the handle 204 and the return cryogen may flow from the needle 202 to the cryogen line 208. The handle 204 may also include a vacuum chamber 226. The vacuum chamber 226 may be positioned at an outer side of the handle 204 and insulate an outer surface of the handle 204 from the extreme temperatures of the cryogen.

In the example shown, the handle 204 is a right-angle handle that transitions from a longitudinal or axial direction of the cryogen line 208 to a longitudinal or axial direction of the needle 202. In this example, the needle 202 is oriented at 90 degrees or perpendicular to the axial direction of the cryogen line 208. In other examples, the needle 202 may be oriented at a different angle from the cryogen line 208. In still other examples, the handle 204 may be flexible to allow adjustment of the relative orientation of the needle 202 from the cryogen line 208.

In existing cryogen assemblies, the handle 204 often includes a heat exchanger positioned inside. Here, the handle 204 does not include a heat exchanger. Instead, a heat exchanger 206 is positioned inside the cryogen line 208 that is coupled to the handle 204. Because the handle 204 does not include a heat exchanger, as is typically needed in Joule-Thompson cryoprobes, the handle 204 can be smaller and/or have less mass than existing cryoprobe handles. The handle 204 in the cryoprobes of the instant disclosure may have a first outer width or diameter d1 at a first portion and may have a second outer width or diameter d2 at a second portion. In some examples, the first diameter d1 and/or the second diameter d2 may be less than 10 mm. In other examples, the first diameter d1 and/or the second diameter d2 may be less than 8 mm. In still other examples, the first diameter d1 and/or the second diameter d2 may be less than 5 mm. Existing cryoprobes do not have such small sizes due to the existence of a heat exchanger. For example, existing cryoprobe handles may weigh in a range of about 15 grams to about 60 grams. The cryoprobe handles of the present disclosure may be reduced from these amounts to have a weight of less than about 15 grams. In some examples, the cryoprobe handles may weigh about 5 grams or less. The cryoprobes of the present disclosure may weigh less than 50% or at least 33% of existing designs.

As shown in FIG. 2, the cryogen line 208 is coupled to the handle 204. The cryogen flow path from the needle 202 and the handle 204 may continue through the cryogen line 208 to allow cryogen to be supplied and then returned to the cryogen source 108. The heat exchanger 206 may be positioned inside the cryogen line 208. The cryogen line 208 may include a cryogen supply 220 that may be a tube, lumen, or other cavity through which cryogen may flow through the cryogen line 208 toward the needle 202. The cryogen line 208 may also include a cryogen return 224 that may be an opening, lumen, flow path, or other cavity through which cryogen may flow from the needle 202. The cryogen supply 220 and the cryogen return 224 are used to move cryogen in directions opposite to each other and create a counter-flow heat exchanger 206.

The heat exchanger 206 may include elements of made of a high thermal conductivity material. Example materials may include copper, brass, or other materials. The heat exchanger 206 may extend along an entire length of the cryogen line 208. In some examples, the cryogen line 208 is about 12 feet in length. In other examples, the cryogen line 208 is at least 10 feet in length. The heat exchanger 206 may have a length (measured along the outer surface of the cryogen line 208) that is 12 feet in length. In other examples, the heat exchanger 206 may have a length of at least 10 feet in length. In other examples, the heat exchanger 206 may have other lengths. The cryogen line 208 and or the heat exchanger 206 may a length that is sized so as to achieve a desired amount of heat transfer and/or to achieve a predetermined temperature at the needle 202. It may be desirable in some instances to include multiple heat exchangers 206 positioned along the cryogen line 208. Such multiple heat exchangers 206 may be separated from each other and be periodically positioned along the length of the cryogen line 208.

The heat exchanger 206 may allow thermal energy from the supply cryogen flowing toward the needle 202 to be transferred to the return cryogen flowing away from the needle 202. In this manner, the temperature of the supply cryogen is lowered before it reaches the tip 228 of the needle 202. Thus, when the supply cryogen expands at the Joule-Thompson chamber, the temperature of the cryogen drops to a lower temperature than it otherwise would reach since it has a lower incoming temperature. The cryogen then flows away from the needle 202 and enters the heat exchanger 206 to cool the then incoming supply cryogen.

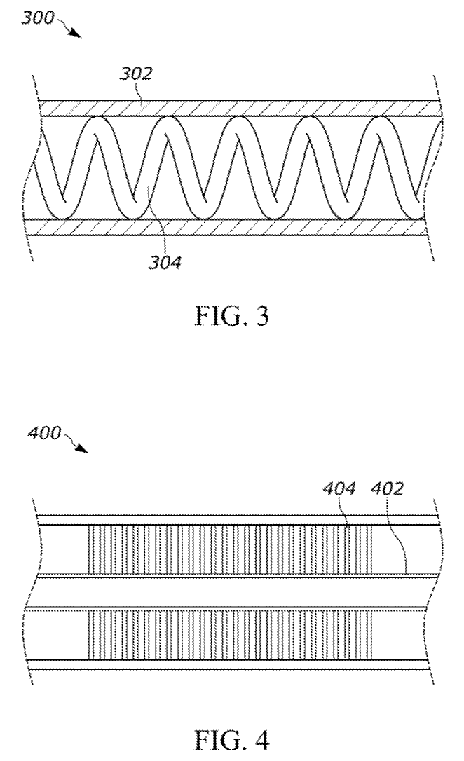

Referring now to FIG. 3, an example heat exchanger 300 is shown. The cryogen line 208 may have the structure shown in the example heat exchanger 300. In this example, the heat exchanger 300 may include an outer tube or shell 302 and an inner helical lumen 304. The supply cryogen may flow toward the needle 202 through the helical lumen 304. The return cryogen may flow away from the needle through the outer tube 302. The helical shape of the helical lumen 304 includes an increased amount of surface area compared to a linear lumen. The increased surface area of the helical lumen 304 allows increased thermal energy to be transferred from the supply cryogen in the helical lumen 304 to the return cryogen flowing around the helical lumen 304 in the outer tube 302. The helical lumen 304 may be made of a material of high thermal conductivity such as copper or brass in some examples.

As shown in FIG. 4, another example heat exchanger 400 may include a structure with a fin and tube configuration. The example heat exchanger 400 may include an inner lumen 402 that is surrounded by or in contact with a series of fins 404. The inner lumen 402 and the fins 404 may be positioned inside an outer shell or tube 406. The supply cryogen may flow toward the needle 202 through the inner lumen 402. The return cryogen may flow away from the needle 202 through the outer shell 406. The return cryogen may flow over and around the inner lumen 402 and the fins 404. The increased surface area of the fins 404 facilitates a transfer of thermal energy from the cryogen in the inner lumen 402 to the return cryogen in the outer shell 406. In some examples, the inner lumen 402 and the fins 404 may be made of material with high thermal conductivity such as copper or brass.

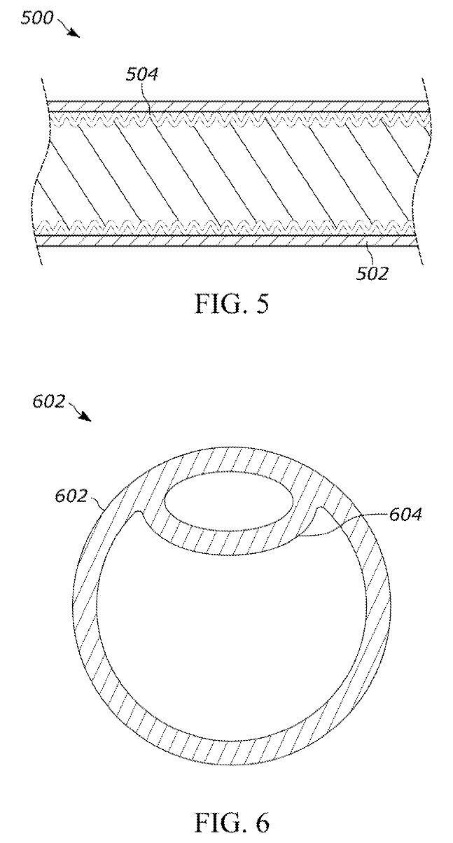

Turning now to FIG. 5, another example heat exchanger 500 is shown. The cryogen line 208 may have a structure such as that of heat exchanger 500. In this example, the heat exchanger 500 includes an outer tube 502 and an inner lumen 504. The inner lumen 504 is positioned inside the outer tube 502 and includes an outer surface with grooves and/or ridges for increased heat transfer. The outer surface of inner lumen 504 may have a helical or spiral configuration. As the return cryogen flows over the outer surface of the inner lumen 504, the return cryogen flows in a helical or spiraling flow pattern. Such increased engagement with the inner lumen 504 causes increased thermal transfer from the supply cryogen in the inner lumen 504 to the return cryogen flowing around the inner lumen 504. The inner lumen 504 may be made of material with high thermal conductivity such as copper or brass.

In yet another example, the cryogen line 208 may have a structure such as that shown in FIG. 6. In this example, the example cryogen line 600 may include an outer wall 602 and an inner wall 604. The inner wall 604 may define a supply lumen through which supply cryogen may flow toward the needle 202. The outer wall 602 and an outer side the inner wall 604 may define a return lumen. This structure may be used to transfer thermal energy from the supply cryogen to the return cryogen.

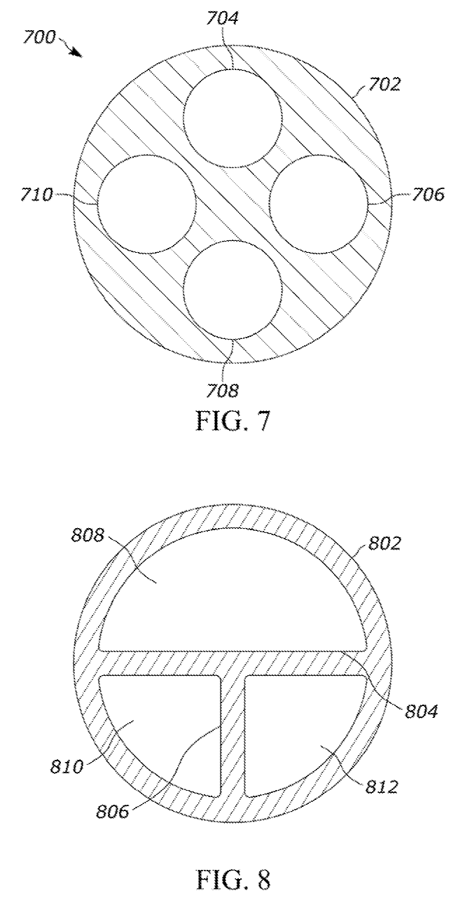

Another example cryogen line 700 is shown in FIG. 7. The cryogen line 208 may have the structure of cryogen line 700. In this example, the cryogen line 700 may include a base 702 through which multiple lumens are positioned. In this example, the base 702 includes a first lumen 704, a second lumen 706, a third lumen 708, and a fourth lumen 710. Supply cryogen may be moved through one or more of the first lumen 704, the second lumen 706, the third lumen 708, and/or the fourth lumen 710. Return cryogen may be moved through one or more lumens that do not supply cryogen to the needle 202. The lumens may be in contact with one another or may be separated from a material with high thermal conductivity to transfer thermal energy from the supply cryogen to the return cryogen. In this example, the cryogen line 700 includes four lumens but in other examples other quantities of lumens may be included.

Another example cryogen line 800 is shown in FIG. 8. The cryogen line 208 may have the structure of cryogen line 800. The cryogen line 800 may include an outer wall 802. The cryogen line 800 may also include a first inner wall 804 and a second inner wall 806. The first inner wall 804 and the second inner wall 806 may divide the inner space inside outer wall 802 into one or more lumens or flow paths. In one example, the supply cryogen may flow through the lumen 812 defined by the outer wall 802, the first wall 802 and the second wall 806. The return cryogen may flow through the lumen 808 defined by the outer wall 802 and the first wall 804. The lumen 810 may be used for other purposes such as for housing wires or other elements of the cryoprobe that may transfer sensor signals or other electrical signals from the cryoprobe to the cryoprobe console or other treatment devices.

The cryoprobe assemblies of the present disclosure with cryogen lines that include heat exchangers demonstrate improved performance over existing cryoprobes. Simulations and testing performed using cryogen lines with heat exchangers show improved performance over existing cryoprobes that do not include the cryogen lines with heat exchangers and include heat exchangers in the handles of the probes. Testing simulations were performed comparing three existing cryoprobes with heat exchangers in the handles to a cryoprobe assembly similar to the cryoprobe assembly 200 shown in FIG. 2.

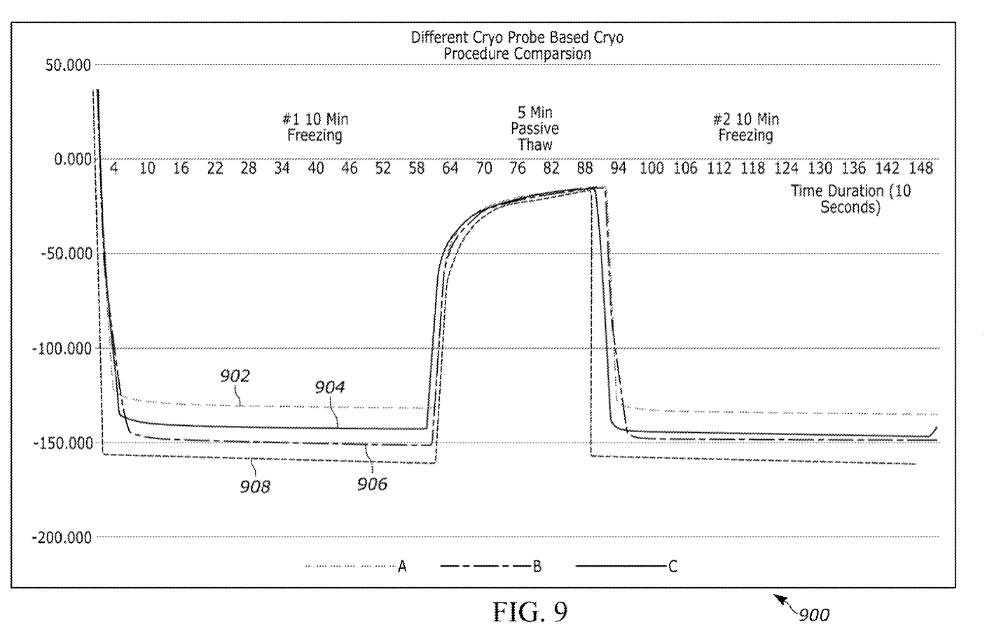

The results of such testing is included in the temperature plot 900 shown in FIG. 9. The graph shows the temperature measured at or near a tip of the needle of the cryoprobe during a simulated cryoablation freezing process. Each of the cryoprobes was operated to perform a first 10 minute freezing cycle followed by a 5 minute passive thaw cycle followed by a second 10 minute freezing cycle. The line 902 indicates the performance of a traditional cryoprobe A with a heat exchanger in the handle. The line 904 indicates the performance of a traditional cryoprobe B with a heat exchanger in the handle. The line 906 indicates the performance of a traditional cryoprobe C with a heat exchanger in the handle. The line 908 indicates the performance of a cryoprobe X that includes a cryogen line such as that previously described with a heat exchanger positioned inside and along a length of the cryogen line. The cryoprobe X did not include a heat exchanger in the handle. As can be seen, the cryoprobe X demonstrates improved performance over all the traditional cryoprobes. The cryoprobe X is able to achieve a target temperature below -150 degrees Celsius more quickly than the other cryoprobes and achieves a lower temperature than the other cryoprobes.

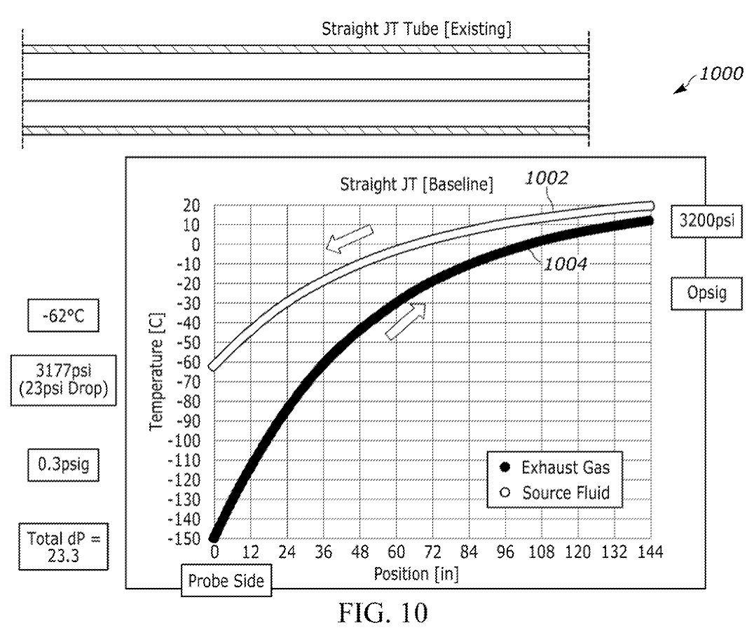

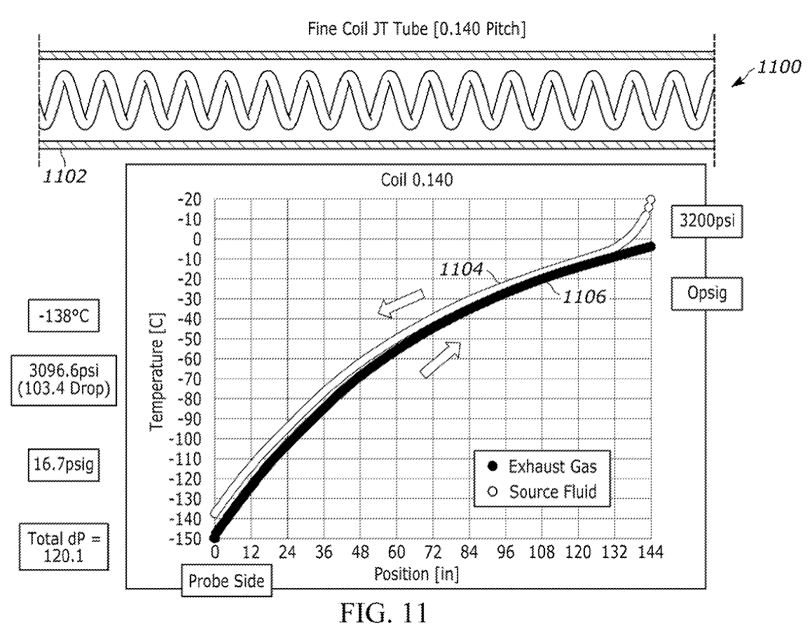

Further testing was performed to compare a performance of a traditional cryogen line with a cryogen line of the present disclosure. FIG. 10 illustrates a performance of a traditional cryogen line and FIG. 11 illustrates a performance of cryogen line that includes a heat exchanger structure to improve the transfer of thermal energy between the supply cryogen and the return cryogen.

Referring first to FIG. 10, a test was performed to determine a temperature of the cryogen at various positions along a length of the cryogen line. In this test, a traditional cryogen line 1010 was used in the test. The cryogen line 1010 includes a traditional structure in which a centrally located supply lumen is positioned inside an outer tube. Supply cryogen flows through the inner supply lumen (in a direction toward the needle of the cryoprobe) and return cryogen flows in an opposite direction in the return path formed by the inner surface of the outer tube and outer surface of the inner lumen. The graph 1000 shows a first plot 1002 that indicates a temperature of the supply cryogen. The x-axis of the graph labelled “position” indicates a position along the cryogen line with position “0” indicating a location at which the cryogen line connects to the cryoprobe and position “144” indicating inches away from the cryoprobe. In this test, the cryogen line was 144 inches (or 12 feet) long. As can be seen, the supply cryogen starts at approximately 20 degrees Celsius and slowly drops to approximately -60 degrees Celsius when it reaches the cryoprobe.

Turning now to FIG. 11, the same test was performed using a cryogen line that includes a heat exchanger for improved thermal transfer. In this example, the cryogen line 1102 includes an inner lumen that has a helical shape as it extends through the outer tube. In this example, the helical inner lumen had a pitch of 0.140. The supply cryogen travels toward the cryoprobe through the helical inner lumen and the return cryogen flows in the opposite direction over and around the helical inner lumen. The graph 1100 shows the same test data for this cryogen line with heat exchanger. As can be seen, the performance of the cryogen line 1102 shows significant improvement over the performance of the traditional cryogen line 1010. As can be seen, the supply cryogen begins at about the same temperature 20 degrees Celsius at position 144. As the supply cryogen moves toward the cryoprobe (position 0), the temperature of the cryogen drops and follows the temperature of the returning cryogen (line 1106). At position 0 in this example, the supply cryogen has reached a temperature of about -138 degrees Celsius which is significantly lower than the temperature of -62 degrees Celsius achieved using the traditional cryogen line.

The following is a list of non-limiting illustrative embodiments disclosed herein:

Illustrative embodiment 1: A cryoprobe assembly may including a needle, a flexible conduit, and a handle portion configured to couple the needle to the flexible conduit to create a cryogen flow path. The flexible conduit including a counterflow heat exchanger.

Illustrative embodiment 2: The cryoprobe assembly of illustrative embodiment 1, wherein the handle portion does not include a heat exchanger.

Illustrative embodiment 3: The cryoprobe assembly of any of illustrative embodiments 1 or 2, wherein the needle defines a Joule-Thompson expansion chamber.

Illustrative embodiment 4: The cryoprobe assembly of any of illustrative embodiments 1 to 3, wherein the counterflow heat exchanger is flexible.

Illustrative embodiment 5: The cryoprobe assembly of any of illustrative embodiments 1 to 4, wherein the counterflow heat exchanger has a length of at least 12 inches.

Illustrative embodiment 6: The cryoprobe assembly of any of illustrative embodiments 1 to 5, wherein the flexible conduit comprises a plurality of counter flow heat exchangers spaced apart from one another.

Illustrative embodiment 7: The cryoprobe assembly of any of illustrative embodiments 1 to 6, wherein the handle portion comprises a vacuum chamber to insulate an exterior of the handle portion from cryogen flow path.

Illustrative embodiment 8: The cryoprobe assembly of any of illustrative embodiments 1 to 7, wherein the counterflow heat exchanger includes a supply path through which cryogen flows toward a distal end of the needle and a return path through which the cryogen flows away from the distal end of the needle, the counterflow heat exchanger configured to transfer thermal energy from the cryogen in the supply path to the cryogen in the return path to lower a temperature of the cryogen in the supply path.

Illustrative embodiment 9: The cryoprobe assembly of any of illustrative embodiments 1 to 8, wherein the counterflow heat exchanger has a predetermined length that causes the distal end of the needle to achieve a temperature of about -140 degrees Celsius or lower.

Illustrative embodiment 10: The cryoprobe assembly of any of illustrative embodiments 1 to 9, wherein the supply path or the return path has a helical shape.

Illustrative embodiment 11: The cryoprobe assembly of any of illustrative embodiments 1 to 9, wherein the counterflow heat exchanger comprises a plurality of fins extend from the supply path into the return path.

Illustrative embodiment 12: The cryoprobe assembly of any of illustrative embodiments 1 to 11, wherein the handle portion is configured to transition from a first axial direction of the flexible conduit to a second axial direction of the needle.

Illustrative embodiment 13: The cryoprobe assembly of illustrative embodiment 12, wherein the second axial direction is offset 90 degrees from the first axial direction.

Illustrative embodiment 14: The cryoprobe assembly of illustrative embodiment 12, wherein the handle portion is adjustable to vary an offset angle between the first axial direction and the second axial direction.

Illustrative embodiment 15: The cryoprobe assembly of any of illustrative embodiments 1 to 14, wherein a maximum width of the handle portion is less than about 10 mm.

Illustrative embodiment 16: The cryoprobe assembly of any of illustrative embodiments 1 to 15, wherein a maximum width of the handle portion is less than about 5 mm.

Illustrative embodiment 17: The cryoprobe assembly of any of illustrative embodiments 1 to 16, wherein a mass of the handle portion is less than about X grams.

Illustrative embodiment 18: A method comprising moving a cryogen through a supply path in a flexible conduit toward a distal end of a needle of a cryoprobe; pre-cooling the cryogen in the flexible conduit prior to reaching the distal end of the needle of the cryoprobe; and allowing the pre-cooled cryogen to expand in a Joule-Thompson expansion chamber at the distal end of the needle to achieve a temperature of about -140 degrees Celsius or lower.

Illustrative embodiment 19: The method of illustrative embodiment 18, wherein the pre-cooling of the cryogen is performed using a counterflow heat exchanger positioned in the flexible conduit.

Illustrative embodiment 20: The method of illustrative embodiment 18 or 19, wherein the step of moving the cryogen through the supply path comprises moving the cryogen through a handle portion of the cryoprobe that connects the flexible conduit to the needle, and the step of pre-cooling the cryogen is performed without a heat exchanger in the handle portion.

Illustrative embodiment 21: A cryoablation system comprising: a cryogen supply apparatus including a pump to supply cryogen from a cryogen source; and a probe assembly comprising: a needle; a handle portion; and a flexible conduit comprising a counterflow heat exchanger coupling the handle portion to the cryogen supply apparatus.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A cryoprobe assembly comprising:
a needle;
a flexible conduit; and
a handle portion configured to couple the needle to the flexible conduit to create a cryogen flow path,
wherein the flexible conduit comprises a counterflow heat exchanger.

2. The cryoprobe assembly of claim 1, wherein the handle portion does not include a heat exchanger.

3. The cryoprobe assembly of claim 1, wherein the needle defines a Joule-Thompson expansion chamber.

4. The cryoprobe assembly of claim 1, wherein the counterflow heat exchanger is flexible.

5. The cryoprobe assembly of claim 1, wherein the counterflow heat exchanger has a length of at least 12 inches.

6. The cryoprobe assembly of claim 1, wherein the flexible conduit comprises a plurality of counter flow heat exchangers spaced apart from one another.

7. The cryoprobe assembly of claim 1, wherein the handle portion comprises a vacuum chamber to insulate an exterior of the handle portion from cryogen flow path.

8. The cryoprobe assembly of claim 1, wherein the counterflow heat exchanger includes a supply path through which cryogen flows toward a distal end of the needle and a return path through which the cryogen flows away from the distal end of the needle, the counterflow heat exchanger configured to transfer thermal energy from the cryogen in the supply path to the cryogen in the return path to lower a temperature of the cryogen in the supply path.

9. The cryoprobe assembly of claim 8, wherein the counterflow heat exchanger has a predetermined length that causes the distal end of the needle to achieve a temperature of about -140 degrees Celsius or lower.

10. The cryoprobe assembly of claim 8, wherein the supply path or the return path has a helical shape.

11. The cryoprobe assembly of claim 8, wherein the counterflow heat exchanger comprises a plurality of fins extend from the supply path into the return path.

12. The cryoprobe assembly of claim 1, wherein the handle portion is configured to transition from a first axial direction of the flexible conduit to a second axial direction of the needle.

13. The cryoprobe assembly of claim 12, wherein the second axial direction is offset 90 degrees from the first axial direction.

14. The cryoprobe assembly of claim 12, wherein the handle portion is adjustable to vary an offset angle between the first axial direction and the second axial direction.

15. The cryoprobe assembly of claim 1, wherein a maximum width of the handle portion is less than about 10 mm.

16. The cryoprobe assembly of claim 1, wherein a maximum width of the handle portion is less than about 5 mm.

17. A cryoablation system comprising:
a cryogen supply apparatus including a pump to supply cryogen from a cryogen source; and
a probe assembly comprising:
a needle;
a handle portion; and
a flexible conduit comprising a counterflow heat exchanger coupling the handle portion to the cryogen supply apparatus.

18. A method comprising:
moving a cryogen through a supply path in a flexible conduit toward a distal end of a needle of a cryoprobe;
pre-cooling the cryogen in the flexible conduit prior to reaching the distal end of the needle of the cryoprobe; and
allowing the pre-cooled cryogen to expand in a Joule-Thompson expansion chamber at the distal end of the needle to achieve a temperature of about -140 degrees Celsius or lower.

19. The method of claim 18, wherein the pre-cooling of the cryogen is performed using a counterflow heat exchanger positioned in the flexible conduit.

20. The method of claim 18, wherein the step of moving the cryogen through the supply path comprises moving the cryogen through a handle portion of the cryoprobe that connects the flexible conduit to the needle, and the step of pre-cooling the cryogen is performed without a heat exchanger in the handle portion.
